(19) United States
(12) Patent Application Publication
(10) Pub. No.: US 2023/0218191 A1
(43) Pub. Date: Jul. 13, 2023

(54) Body Composition Measuring Device Using Nine Segments and Operation Method Thereof

(71) Applicant: INBODY CO., LTD., Seoul (KR)

(72) Inventor: Ki Chul Cha, Seoul (KR)

(21) Appl. No.: 17/928,376

(22) PCT Filed: Jul. 8, 2021
(86) PCT No.: PCT/KR2021/008705

§ 371 Date: Nov. 29, 2022

(30) Foreign Application Priority Data
Jul. 8, 2020 (KR) 10-2020-0084255
Jul. 6, 2021 (KR) 10-2021-0088390

(51) Int. Cl. A61B 5/0537; A61B 5/00

(52) U.S. Cl. CPC A61B 5/0537; A61B 5/4878; A61B 5/6891

(57) ABSTRACT

Disclosed are a body composition measuring device using nine segments and an operation method thereof. The disclosed body composition measuring device comprises: both hand electrode parts and both foot electrode parts, each of which has a plurality of electrodes; two arm electrode parts attached to elbow joint regions of both arms, respectively; two leg electrode parts attached to knee joint regions of both legs, respectively; and a processing unit for causing electric current to flow through different combinations of the both hand electrode parts, the both foot electrode parts, the arm electrode parts, and the leg electrode parts at the time of measurement so as to measure impedance values of a measurement object, and then analyzing the body composition of the measurement object.

### TECHNICAL FIELD

The following description relates to a body composition measuring device using nine segments and an operation method thereof.

### BACKGROUND ART

In general, body composition analysis may be used to represent proportions of water, fat, bone, muscle, and the like, in the human body. Information about body composition analysis may be variously used. For example, a person who desires to work out may use information about body composition to make a work-out plan. An obese person may use information about body composition to set a diet goal. A physician may use information about body composition to treat patients. One method of analyzing body composition may be to measure an electrical impedance value of the body. Electrodes may be attached to various parts of the body for a body composition analysis.

### DISCLOSURE OF THE INVENTION

### Technical Solutions

A body composition measuring device according to an example embodiment may include: both hand electrode parts and both foot electrode parts, each of which has a plurality of electrodes;

two arm electrode parts attached to elbow joint regions of both arms, respectively; two leg electrode parts attached to knee joint regions of both legs, respectively; and a processing unit for causing electric current to flow through different combinations of the both hand electrode parts, the both foot electrode parts, the arm electrode parts, and the leg electrode parts at the time of measurement so as to measure impedance values of a measurement object, and then analyzing the body composition of the measurement object.

In the body composition measuring device according to an example embodiment, the processing unit may cause electric current to flow through different combinations of the both hand electrode parts, the both foot electrode parts, the arm electrode parts, and the leg electrode parts, divide the measurement object into nine segments of an upper left arm (ULA), a lower left arm (LLA), a upper right arm (URA), a lower right arm (LRA), a trunk (TR), an upper left leg (ULL), a lower left leg (LLL), an upper right leg (URL), and a lower right leg (LRL), and measure the impedance values of the segments.

In the body composition measuring device according to an example embodiment, the processing unit may apply the electric current to first electrodes included in one pair among the both hand electrode parts, the both foot electrode parts, the arm electrode parts, and the leg electrode parts for a body composition measurement, obtain a voltage for the electric current from second electrodes included in the pair, and measure an impedance value of a segment between the pair by using the electric current and the voltage.

In the body composition measuring device according to an example embodiment, the processing unit may determine whether there is a segment determined to have edema among the segments, based on a change in each impedance value of the segments.

In the body composition measuring device according to an example embodiment, at least some of the arm electrode parts and the leg electrode parts may include an adsorption plate adsorbing to an attachment part and at least one electrode in contact with the attachment part, inside the adsorption plate.

In the body composition measuring device according to an example embodiment, the at least some of the arm electrode parts and the leg electrode parts may further include a pressure regulator configured to lower air pressure in a space between the adsorption plate, which adsorbs to the attachment part, and the attachment part.

In the body composition measuring device according to an example embodiment, at least some of the arm electrode parts and the leg electrode parts may include at least one electrode in contact with the attachment part and a sheet surrounding a periphery of the attachment part to maintain the contact.

In the body composition measuring device according to an example embodiment, at least some of the arm electrode parts and the leg electrode parts may include an electrode board configured to adjust a frequency of the electric current.

In the body composition measuring device according to an example embodiment, the body composition measuring device may be in a form of a chair, the arm electrode parts may be disposed on arm rests of the chair, and the leg electrode parts may be disposed on parts of the chair in contact with knees.

An operation method of a body composition measuring device according to an example embodiment may include: measuring an impedance value of a measurement object by causing electric current to flow through different combinations of both hand electrode parts and both foot electrode parts, each of which has a plurality of electrodes, two arm electrode parts attached to elbow joint regions of arms, respectively, and two leg electrode parts attached to knee joint regions of legs, respectively; and analyzing body composition of the measurement object, based on the impedance value of the measurement object.

In the operation method of the body composition measuring device according to an example embodiment, the measuring of the impedance value of the measurement object may include causing electric current to flow through different combinations of the both hand electrode parts, the both foot electrode parts, the arm electrode parts, and the leg electrode parts, dividing the measurement object into nine segments of an upper left arm (ULA), a lower left arm (LLA), a upper right arm (URA), a lower right arm (LRA), a trunk (TR), an upper left leg (ULL), a lower left leg (LLL), an upper right leg (URL), and a lower right leg (LRL), and measuring the impedance values of the segments.

In the operation method of the body composition measuring device according to an example embodiment, the measuring of the impedance value of the measurement object may include applying the electric current to first electrodes included in one pair among the both hand electrode parts, the both foot electrode parts, the arm electrode parts, and the leg electrode parts for a body composition measurement, obtaining a voltage for the electric current from second electrodes included in the pair, and measuring an impedance value of a segment positioned between the pair by using the electric current and the voltage.

The operation method of the body composition measuring device according to an example embodiment may further include determining whether there is a segment determined to have edema among the segments based on a change in each impedance value of the segments. In operation method of the body composition measuring device according to an example embodiment, at least some of the arm electrode parts and the leg electrode parts may include an adsorption plate adsorbing to an attachment part and at least one electrode in contact with the attachment part, inside the adsorption plate.

In operation method of the body composition measuring device according to an example embodiment, the at least some of the arm electrode parts and the leg electrode parts further may include a pressure regulator configured to lower air pressure in a space between the adsorption plate, which adsorbs to the attachment part, and the attachment part.

In operation method of the body composition measuring device according to an example embodiment, the at least some of the arm electrode parts and the leg electrode parts may include at least one electrode in contact with the attachment part and a sheet surrounding a periphery of the attachment part to maintain the contact.

In operation method of the body composition measuring device according to an example embodiment, the body composition measuring device may be in the form of a chair, the arm electrode parts may be disposed on arm rests of the chair, and the leg electrode parts may be disposed on a part of the chair in contact with the knees.

### EFFECTS

According to an example embodiment, body composition for detailed parts may be measured with a high accuracy by dividing a measurement object into nine segments through different combinations of hand electrode parts, foot electrode parts, arm electrode parts, the leg electrode parts and measuring impedance values of the segments.

According to an example embodiment, edema, which is difficult to detect due to its slow development, may be precisely predicted and diagnosed by dividing a measurement object into nine segments and measuring impedance values of the segments and body composition.

### BRIEF DESCRIPTION OF DRAWINGS

FIGS. 1 and 2 are diagrams illustrating a body composition measuring device according to an example embodiment.

FIGS. 3 through 8 are diagrams illustrating arm electrode parts and leg electrode parts, according to an example embodiment.

FIG. 9 is a diagram illustrating a hand electrode part according to an example embodiment.

FIG. 10 is a diagram illustrating a foot electrode part according to an example embodiment.

FIG. 11 is a diagram illustrating a chair-type body composition measuring device according to an example embodiment.

FIG. 12 is a diagram illustrating a method of operating a body composition measuring device, according to an example embodiment.

### BEST MODE FOR CARRYING OUT THE INVENTION

The following detailed structural or functional description is provided as an example only and various alterations and modifications may be made to the examples. Here, examples are not construed as limited to the disclosure and should be understood to include all changes, equivalents, and replacements within the idea and the technical scope of the disclosure.

Terms, such as first, second, and the like, may be used herein to describe various components. Each of these terminologies is not used to define an essence, order or sequence of a corresponding component but used merely to distinguish the corresponding component from other component(s). For example, a first component may be referred to as a second component, and similarly the second component may also be referred to as the first component.

It should be noted that if it is described that one component is “connected”, “coupled”, or “joined” to another component, a third component may be “connected”, “coupled”, and “joined” between the first and second components, although the first component may be directly connected, coupled, or joined to the second component.

The singular forms “a”, “an”, and “the” are intended to include the plural forms as well, unless the context clearly indicates otherwise. It will be further understood that the terms “comprises/including” and/or “includes/including” when used herein, specify the presence of stated features, integers, steps, operations, elements, and/or components, but do not preclude the presence or addition of one or more other features, integers, steps, operations, elements, components and/or groups thereof.

Unless otherwise defined, all terms, including technical and scientific terms, used herein have the same meaning as commonly understood by one of ordinary skill in the art to which this disclosure pertains. Terms, such as those defined in commonly used dictionaries, are to be interpreted as having a meaning that is consistent with their meaning in the context of the relevant art, and are not to be interpreted in an idealized or overly formal sense unless expressly so defined herein.

Hereinafter, examples will be described in detail with reference to the accompanying drawings. When describing the example embodiments with reference to the accompanying drawings, like reference numerals refer to like constituent elements and a repeated description related thereto will be omitted.

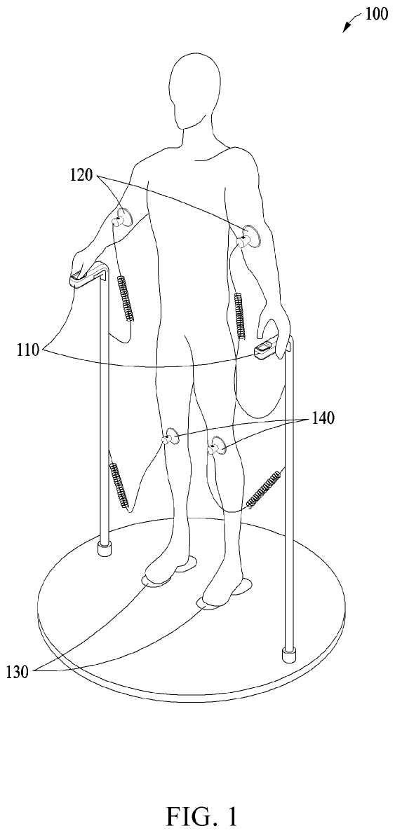

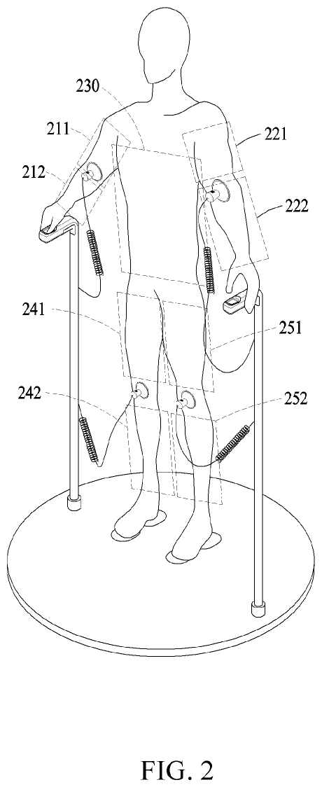

FIGS. 1 and 2 are diagrams illustrating a body composition measuring device according to an example embodiment.

Referring to FIG. 1, a body composition measuring device 100 may include hand electrode parts 110, arm electrode parts 120, foot electrode parts 130, leg electrode parts 140, and a processing unit (not shown).

Body composition analysis or body composition measurement may refer to analyzing elements of a subject's body or a ratio between the elements of the subject's body, with a physical quantity obtained from the subject's body. Measurement may refer to obtaining a physical quantity with a measuring device. Measurement may also include processing a physical quantity to obtain another kind of physical quantity. Therefore, measurement may include applying current to two points of the body and obtaining a voltage difference with a voltmeter and furthermore, calculating an impedance value of two points of the body, based on the applied current and the voltage difference.

Extremities or limbs may be a collective expression of the arms and legs of a human and the four legs of an animal, and the term ‘limb’ may be used to express each of the arms and legs of the human or the animal. The limb may be an arm or a leg. In the case of animals, the limb may be one of the four legs. In the case of humans, an arm may refer to a part extending from a trunk to a hand and may generally include a wrist and a hand. A leg may refer to a part extending is from a trunk to a foot and may generally include an ankle and a foot.

In an example embodiment, in order to analyze body composition, an impedance value of a part of the body may be obtained. Body composition may be analyzed by inputting the obtained impedance value of the body to a predetermined formula along with other measured values (e.g., a height, a weight, and the like). In an example embodiment, other information (e.g., a gender, an age, and others) may also be input to the formula to analyze the body composition. In another example embodiment, the body composition may be analyzed by calculating a body composition ratio corresponding to an impedance value and other measured values in a look-up table that represents a relationship between the body composition and the measured values.

In various example embodiments, two current electrodes and two voltage electrodes may be used to obtain an impedance value (or an electrical resistance value). For example, one current electrode connected to an electric current supply device (not shown) may be in contact with a subject's right hand and the other may be in contact with the subject's right foot. One voltage electrode connected to a voltage measuring device (not shown) may be in contact with the subject's right hand and the other may be in contact with the subject's right foot. An electric current supply device may supply current through the two current electrodes, and the voltage measuring device may measure a voltage difference between the two voltage electrodes. An impedance value of the right arm-trunk-right leg positioned between the electrodes may be calculated from the amount of the applied current and the voltage difference. A simplest formula for calculating an impedance value may be, for example, dividing a voltage difference by an electric current.

Each of the hand electrode parts 110 and the foot electrode parts 130 illustrated in FIG. 1 may include one current electrode and one voltage electrode. Where these electrodes are used, an impedance value of the left arm-trunk-left leg, an impedance value of the left arm-trunk-right arm, an impedance value of the left leg-trunk-right leg, an impedance value of the left arm-body-trunk-right leg, and an impedance value of the right arm-trunk-left leg may be calculated. In such a manner of using various impedance values obtained by differentiating a combination of the electrodes in contact with the body, each of impedance values of the arms, legs, and torso may be calculated.

Furthermore, the arm electrode parts 120 and the leg electrode parts 140 may be additionally used for calculating an impedance value. Each of the arm electrode parts 120 and the leg electrode parts 140 may include one electrode.

The arm electrode parts 120 may be in contact with an elbow joint region of the arm. The arm electrode parts 120 may be in contact with the inside or outside of the elbow. The arm electrode parts 120 attached to the middle of the arm may subdivide the arm part for which the impedance value is calculated. For example, an impedance value of the lower arm between a hand electrode part and an arm electrode part and an impedance value of the upper arm between the arm electrode part and the torso may be calculated separately from each other.

Also, the leg electrode parts 140 may be in contact with a knee joint region of the leg. The leg electrode parts 140 may be in contact with the front or back of the knee. By attaching the leg electrode parts 140 to the middle of the legs, the leg part for which an impedance value is calculated may be subdivided. For example, an impedance value of the lower leg between a foot electrode part and a leg electrode part and an impedance value of the upper leg between the leg electrode part and the torso may be calculated separately.

Referring to FIG. 2, described is a segment for which an impedance value is to be calculated. Nine segments may be set based on electrodes attached to the body. Segments may include an upper left arm (ULA) 211, a lower left arm (LLA) 212, an upper right arm (URA) 221, a lower right arm (LRA) 222, a trunk (TR) 230, an upper left leg (ULL) 241, a lower left leg (LLL) 242, an upper right leg (URL) 251, and a lower right leg (LRL) 252. The segments, that is, the ULA 211, the LLA 212, the URA 221, the LRA 222, the ULL 241, the LLL 242, the URL 251 and the LRL 252, set in the arms and legs may be divided by the arm electrode parts 120 and the leg electrode parts 140. In this way, detailed impedance values of the arms and legs may be calculated through the arm electrode parts 120 and the leg electrode parts 140.

It may be important to check conditions of the body by measuring body composition on a regular basis (e.g., daily, every other day, weekly, and the like). A change in body composition may be examined to determine a change in the body, such as an increase or decrease in water in the body of a patient. A doctor may also determine progression or alleviation of a disease by reviewing a trend of a body composition analysis result. In order to repeat measurement of body composition, a process of attaching an electrode to a subject and removing the electrode from the body of the subject after the measurement may need to be repeated. Where an electrode is attached to the body for a subsequent measurement of body composition, the electrode may need to be attached to the same position as the position of the electrode attached in a previous measurement. In general, since an impedance value of the body may vary depending on a position of an electrode, repeatability of an impedance value may not be expected, where the electrode is attached at a position different from the position where the electrode has been attached is in a previous measurement. The electrode may need to be attached to a consistent position to increase reproducibility. The electrode may be attached to the consistent position by leaving a mark on the body in advance or based on a specific part of the body that may serve as a reference point or a reference mark, for attaching the electrode to the consistent position.

Edema may refer to a state in which fluid, such as lymph fluid or tissue exudate, accumulates and is excessively present in the tissue. Where edema occurs in a part of the body (e.g., a lower leg), an impedance value of the part may change, which may lead to a change in body composition (e.g., change in the amount of water). Accordingly, analysis on body composition may enable determination on a possibility and/or an occurrence of edema.

Since an impedance value of a part of the body is correlated with the amount of water in a part of the body, the impedance value of the part of the body may be an index for determining edema. For example, where an impedance value of a specific part of the body is less than a preset threshold value for the specific part of the body, it may be determined that there is a possibility of edema.

Alternatively, edema of a specific part may be determined based on a change in an impedance value of the specific part of the body. For example, where an impedance value of a specific part of the body remains at a constant level and then the impedance value decreases significantly after a specific time, it may be determined that there is a possibility of edema.

Alternatively, edema may be caused by an increase in extracellular water rather than a change in intracellular water. An impedance value of a part of the body for each electrical signal may be calculated by measuring a voltage difference for each electrical signal by applying a low-frequency electrical signal (e.g., a 5 kHz electrical signal) and a high-frequency electrical signal (e.g., a 500 kHz electrical signal), using a feature that the high-frequency electrical signal passes through a cell wall better than the low-frequency electrical signal. For example, it may be determined that there is edema, where an impedance value measured with the low-frequency electrical signal is different, by a certain value or more, from an impedance value measured with the high-frequency electrical signal. In addition, edema may be determined based on evaluation of a change in the impedance value of the part measured by the high-frequency electrical signal and a change in the impedance value of the part measured by the low-frequency electrical signal. For example, it may be determined that there is edema, where a change rate of the impedance value of the part measured with the low-frequency electrical signal is greater, by a preset range or more, than a change rate of the impedance value of the part measured with the high-frequency electrical signal.

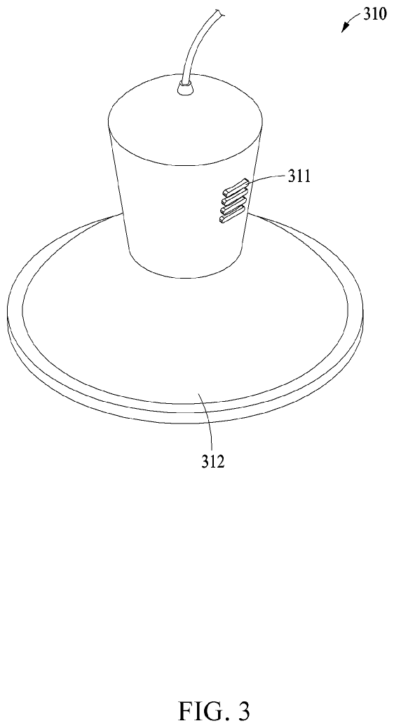

FIGS. 3 to 8 are diagrams illustrating electrode parts and leg electrode parts, according to an example embodiment.

Various structures for attaching an arm electrode part and a leg electrode part to an elbow joint region and a knee joint region, respectively, may be applicable.

Referring to FIG. 3, an electrode part 310 may be an arm electrode part attached to an elbow joint region in an arm or a leg electrode part attached to a knee joint region in a leg. Where, in the electrode part 310, an adsorber 312 contacts an elbow joint region or a knee joint region while a handle portion 311 is pressed like a dropper, pressure may increase between the adsorber 312 and a part of the body in contact and thus the electrode part 310 may be attached to the part of the body in contact. In this case, since a cable connected to the electrode part 310 is designed to have a light weight, the electrode part 310 may not be detached, due to the weight, from the part of the body in contact.

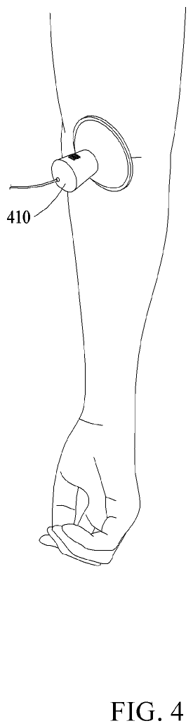

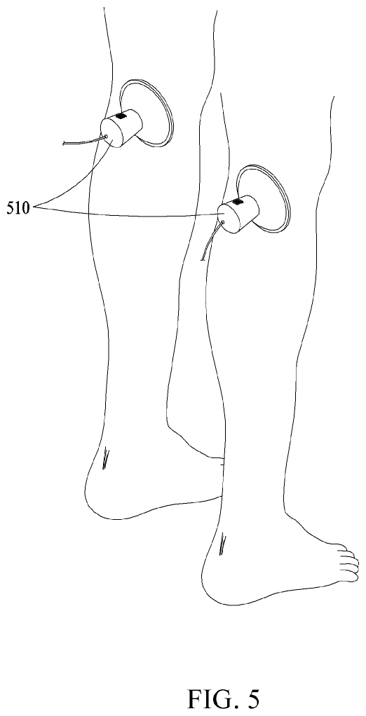

Referring to FIG. 4, an arm electrode part 410 attached to an elbow joint region is illustrated as an example. Referring to FIG. 5, a leg electrode part 510 attached to a knee joint region is illustrated as an example.

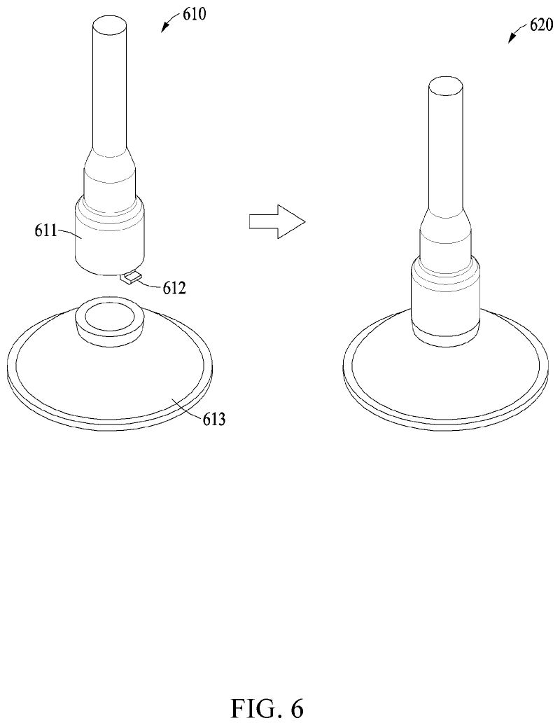

Referring to FIG. 6, electrode parts 610 and 620 may be designed to be replaceable. The electrode part 610 may indicate a state in which an electrode part body 611 and an adsorber 613 are separated from each other, and the electrode part 620 may indicate a state in which the electrode part body 611 and the adsorber 613 are coupled to each other.

In the electrode parts 610 and 620, the electrode part body 611 and the adsorber 613 may be separated from each other, and the electrode parts 610 and 620 may include an electrode 612 in direct contact with a subject. Unlike the electrode part 310 of FIG. 3, the electrode parts 610 and 620 may effectively prevent an adsorptive force of the adsorber 613 from weakening due to continuous use since the adsorber 613 may be detachable and replaceable. The electrode parts 610 and 620 may be implemented by changing all of the rest except for the adsorber 613 into two shot injection molding inside the cable.

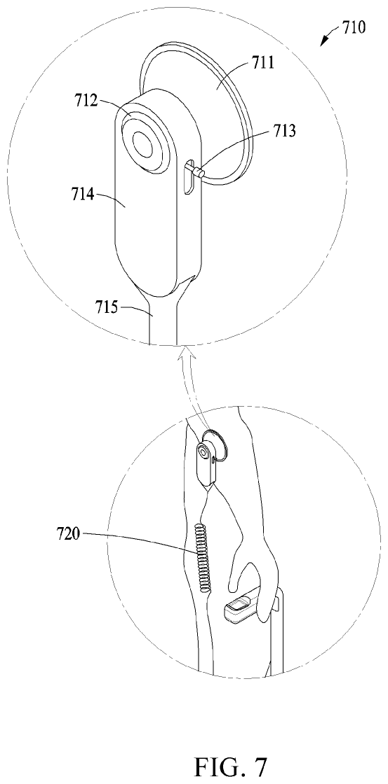

Referring to FIG. 7, an electrode part 710 may be attached to the body of a subject through electronic decompression. The electrode part 710 may include an adsorber 711, a pump button 712, a lock button 713, an electrode board 714, and a Jabara 715. The electrode part 710 may be electrically connected to another component (e.g., an electric current supply device) through a curl cable.

The adsorber 711 may be a part attached to the body of a subject, and the pump button 712 may be a button controlling on/off of electronic decompression. The lock button 713 may control an operation of the pump button 712. For example, the pump button 712 may not operate where the lock button 713 is on.

The electrode board 714 may control a frequency of electric current applied to the subject through the electrode part 710. The current supply device described above may sequentially apply electric current having a different frequency. A voltage measuring device may measure a voltage difference between two electrodes in a state in which electric current having a different frequency is sequentially applied. The frequency of the sequentially applied current may be, for example, 1 kHz, 5 kHz, 50 kHz, 250 kHz, or 500 kHz.

An impedance value for each frequency may be calculated by processing a voltage signal for each frequency measured by the voltage measuring device. The body composition of the subject may be analyzed by applying an impedance value for each frequency to a predetermined formula or table. In general, a high-frequency electrical signal may pass through a cell wall better than a low-frequency electrical signal. An impedance value measured when the high-frequency current is applied may reflect the amount of water in the cell. Accordingly, a body composition analysis result using all of the impedance values for each frequency may more accurately reflect the actual body composition of a subject.

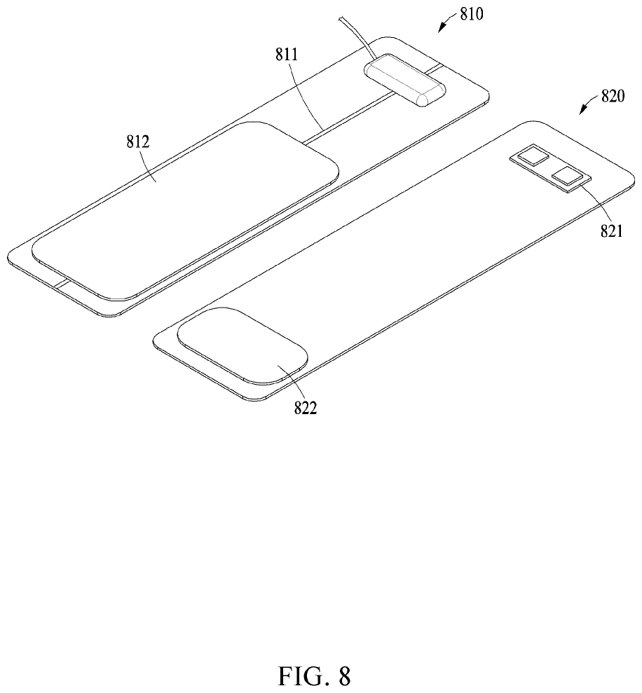

Referring to FIG. 8, electrode parts 810 and 820 may be designed in a cuff shape to wind around an elbow joint region or a knee joint region of a subject. The electrode part 810 may represent an outer surface at the time of winding around the body part in contact, and the electrode part 820 may represent an inner surface at the time of winding around the body part contacted. Through a guideline 811 drawn on the sheet of the electrode parts 810 and 820, a subject or a doctor may wind around a part in contact with the electrode parts 810 and 820 in a pre-intentioned state. When the electrode parts 810 and 820 are wound around the body part contacted, a first velcro 812 may be attached to a second velcro 822, so that the electrode parts 810 and 820 may be fixed to the body part contacted. In this case, the electrode 821 may be in direct contact with the body part contacted.

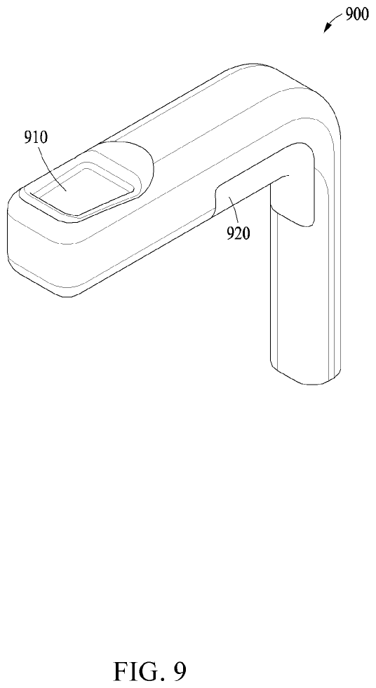

FIG. 9 is a diagram illustrating a hand electrode part.

Referring to FIG. 9, a hand electrode part 900 may include a first electrode 910 and a second electrode 920. Where a subject grips the hand electrode part 900, due to the arrangement of the first electrode 910 and the second electrode 920, the first electrode 910 may naturally come into contact with a subject's thumb, and the second electrode 920 may be in contact with the remaining fingers of the subject.

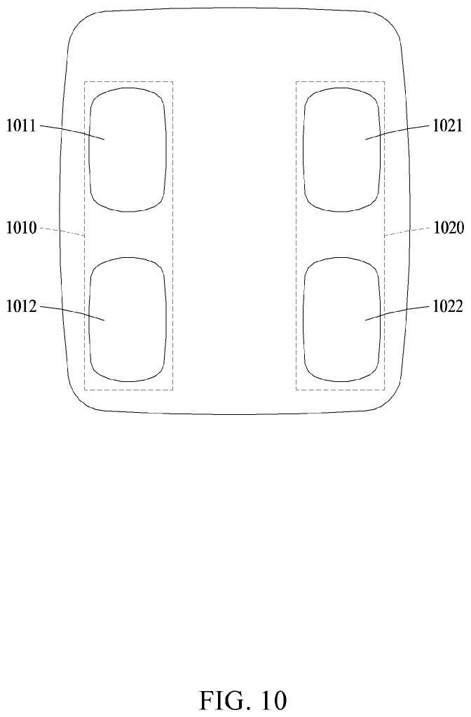

FIG. 10 is a diagram illustrating a foot electrode part according to an example embodiment.

Referring to FIG. 10, foot electrode parts 1010 and 1020 may include first electrodes 1011 and 1021 and second electrodes 1012 and 1022, respectively. Due to the arrangement of the first electrodes 1011 and 1021 and the second electrodes 1012 and 1022, when a subject stands on the foot electrode parts 1010 and 1020, the first electrodes 1011 and 1021 may be naturally in contact with the subject's tips of the toes, and the second electrodes 1012 and 1022 may be in contact with the subject's heels.

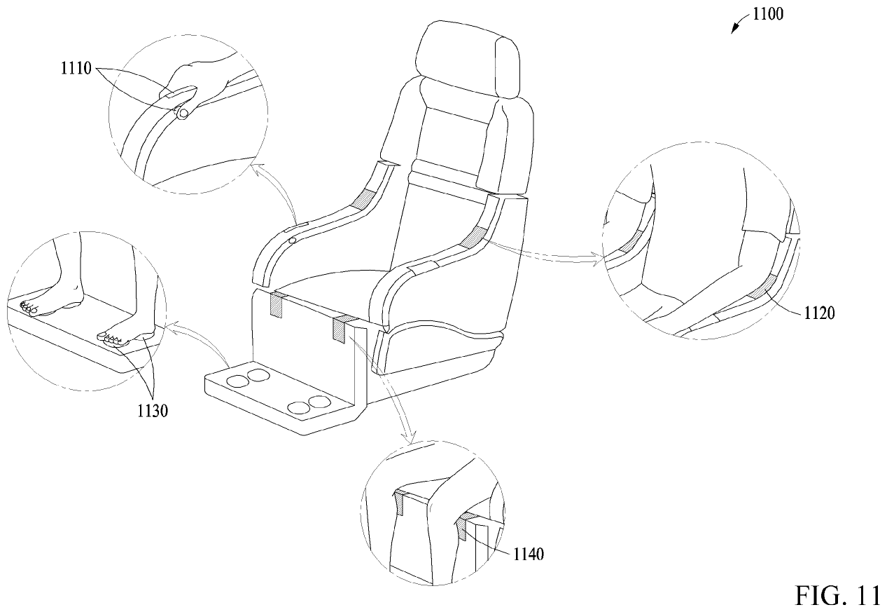

FIG. 11 is a diagram illustrating a chair-type body composition measuring device according to an example embodiment.

Referring to FIG. 11, a chair-type body composition measuring device 1100 may include two hand electrode parts 1110, two arm electrode parts 1120, two foot electrode parts 1130, and two leg electrode parts 1140. The hand electrode parts 1110 may be disposed on a part on which hands are placed, the arm electrode parts 1120 may be disposed on arm rests, the foot electrode parts 1130 may be disposed on footrests of a chair or the floor, and the leg electrode parts 1140 may be placed on a part of the chair in contact with the knees. Where a subject sits on the chair-type body composition measuring device 1100, each electrode part may naturally contact a body part corresponding to each electrode part. Where the subject sits on the chair-type body composition measuring device 1100, natural contact with electrodes and a high reproducibility may be induced. The chair-type body composition measuring device 1100 may be implemented with, for example, a massage chair or a chair installed in a moving means (e.g., a vehicle, an airplane, and the like).

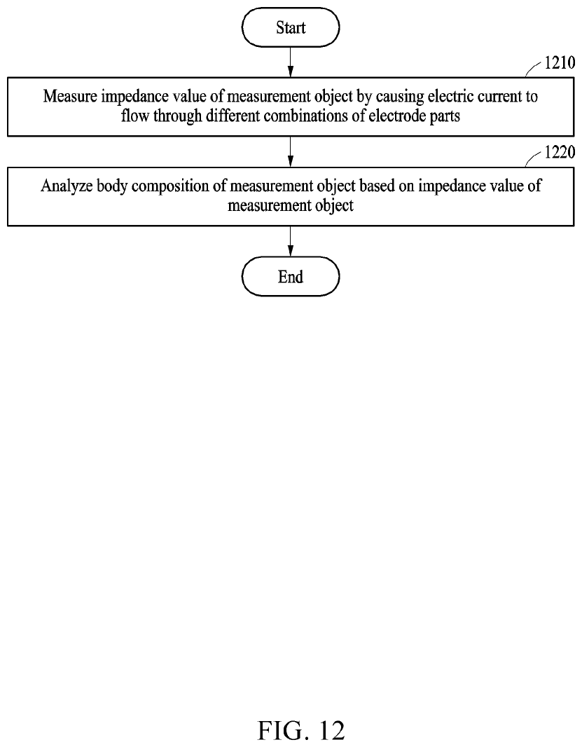

FIG. 12 is a diagram illustrating an operation method of a body composition measuring device according to an example embodiment.

Referring to FIG. 12, an operation method performed by a body composition measuring device is illustrated.

In operation 1210, the body composition measuring device may measure an impedance value of a measurement object by causing an electric current to flow through different combinations of both hand electrode parts and both foot electrode parts, each of which has a plurality of electrodes, two arm electrode parts attached to elbow joint regions of both arms, respectively, and two leg electrode parts attached to knee joint regions of legs, respectively. The body composition measuring device may cause an electric current to flow through different combinations of the both hand electrode parts, the both foot electrode parts, the arm electrode parts, and the leg electrode parts, so that the measurement object may be divided into nine segments of ULA, LLA, URA, LRA, TR, ULL, LLL, URL, and LRL to measure impedance values. The body composition measuring device may apply electric current to first electrodes included in one pair among the both hand electrode parts, the both foot electrode parts, the arm electrode parts, and the leg electrode parts, for body composition measurement, and may obtain a voltage for the current from second electrodes included in the one pair and measure an impedance value of a segment between the one pair by using the current and the voltage. In the embodiment, described above, in which the impedance value of the right arm-trunk-right leg is calculated, the body composition measuring device may select a pair of a right hand electrode part and a right foot electrode part, apply electric current to a first electrode, which is one of the two electrodes included in the right hand electrode part and the left foot electrode part, respectively, for the purpose of body composition measurement, obtain a voltage of the current from second electrode, which is the other of the two electrodes included in the right hand electrode part and the right foot electrode part, respectively, and then measure an impedance value of the segment between the right hand electrode part and the right foot electrode part, that is, the impedance value of the right arm-trunk-right leg, by using the current and the voltage. Herein, for convenience of description, a subject may also be referred to as a measurement object.

At least some of the arm electrode parts and the leg electrode parts may include an adsorption plate that adsorbs to an attachment part and at least one electrode that contacts the attachment part inside the adsorption plate. In addition, at least some of the arm electrode parts and the leg electrode parts may further include a pressure regulator that decreases air pressure in a space between the attachment part and the adsorption plate that adsorbs to the attachment part. Alternatively, at least some of the arm electrode parts and the leg electrode parts may include a sheet surrounding the periphery of the attachment part to maintain contact with at least one electrode in contact with the attachment part.

In operation 1220, the body composition measuring device may analyze the body composition of the measurement object, based on the impedance value of the measurement object.

The body composition measuring device may determine whether there is a segment having edema among segments, based on a change in the impedance value of each of the segments.

The body composition measuring device may be in the form of a chair, the arm electrodes is may be disposed on the arm rests of the chair, and the leg electrodes may be disposed on a part of the chair in contact with the knees.

For a more detailed description of the operations described above with reference to FIG. 12, reference may be made to what is described above with reference to FIGS. 1 through 11.

The examples described herein may be implemented using a hardware component, a software component and/or a combination thereof. A processing device may be implemented using one or more general-purpose or special-purpose computers, such as, for example, a processor, a controller and an arithmetic logic unit (ALU), a digital signal processor (DSP), a microcomputer, an FPGA, a programmable logic unit (PLU), a microprocessor or any other device capable of responding to and executing instructions in a defined manner. The processing device may run an operating system (OS) and one or more software applications that run on the OS. The processing device also may access, store, manipulate, process, and create data in response to execution of the software. For purpose of simplicity, the description of a processing device is used as singular; however, one skilled in the art will appreciate that a processing device may include multiple processing elements and multiple types of processing elements. For example, the processing device may include a plurality of processors, or a single processor and a single controller. In addition, different processing configurations are possible, such as parallel processors.

The software may include a computer program, a piece of code, an instruction, or some combination thereof, to independently or uniformly instruct or configure the processing device to operate as desired. Software and data may be embodied permanently or temporarily in any type of machine, component, physical or virtual equipment, computer storage medium or device, or in a propagated signal wave capable of providing instructions or data to or being interpreted by the processing device. The software also may be distributed over network-coupled computer systems so that the software is stored and executed in a distributed fashion. The software and data may be stored by one or more non-transitory computer-readable recording mediums.

The methods according to the above-described examples may be recorded in non-transitory computer-readable media including program instructions to implement various operations of the above-described examples. The media may also include, alone or in combination with the program instructions, data files, data structures, and the like. The program instructions recorded on the media may be those specially designed and constructed for the purposes of examples, or they may be of the kind well-known and available to those having skill in the computer software arts. Examples of non-transitory computer-readable media include magnetic media such as hard disks, floppy disks, and magnetic tape; optical media such as CD-ROM discs, DVDs, and/or Blue-ray discs; magneto-optical media such as optical discs; and hardware devices that are specially configured to store and perform program instructions, such as read-only memory (ROM), random access memory (RAM), flash memory (e.g., USB flash drives, memory cards, memory sticks, etc.), and the like. Examples of program instructions include both machine code, such as produced by a compiler, and files containing higher-level code that may be executed by the computer using an interpreter.

The above-described devices may be configured to act as one or more software modules in order to perform the operations of the above-described examples, or vice versa.

As described above, although the examples have been described with reference to the limited drawings, a person skilled in the art may apply various technical modifications and variations based thereon. For example, suitable results may be achieved if the described techniques are performed in a different order and/or if components in a described system, architecture, device, or circuit are combined in a different manner and/or replaced or supplemented by other components or their equivalents.

Accordingly, other implementations are within the scope of the following claims.

1. A body composition measuring device comprising:
both hand electrode parts and both foot electrode parts, each of which has a plurality of electrodes;
two arm electrode parts attached to elbow joint regions of both arms, respectively;
two leg electrode parts attached to knee joint regions of both legs, respectively; and
a processing unit for causing electric current to flow through different combinations of the both hand electrode parts, the both foot electrode parts, the arm electrode parts, and the leg electrode parts at the time of measurement so as to measure impedance values of a measurement object, and then analyzing the body composition of the measurement object.

2. The body composition measuring device of claim 1, wherein the processing unit is configured to cause electric current to flow through different combinations of the both hand electrode parts, the both foot electrode parts, the arm electrode parts, and the leg electrode parts, divide the measurement object into nine segments of an upper left arm (ULA), a lower left arm (LLA), a upper right arm (URA), a lower right arm (LRA), a trunk (TR), an upper left leg (ULL), a lower left leg (LLL), an upper right leg (URL), and a lower right leg (LRL), and measure impedance values of the segments.

3. The body composition measuring device of claim 2, wherein the processing unit is configured to apply the electric current to first electrodes comprised in one pair among the both hand electrode parts, the both foot electrode parts, the arm electrode parts, and the leg electrode parts for a body composition measurement, obtain a voltage for the electric current from second electrodes comprised in the pair, and measure an impedance value of a segment between the pair by using the electric current and the voltage.

4. The body composition measuring device of claim 2, wherein the processing unit is configured to determine whether there is a segment determined to have edema among the segments, based on a change in each impedance value of the segments.

5. The body composition measuring device of claim 1, wherein at least some of the arm electrode parts and the leg electrode parts comprises an adsorption plate adsorbing to an attachment part and at least one electrode in contact with the attachment part inside the adsorption plate.

6. The body composition measuring device of claim 5, wherein the at least some of the arm electrode parts and the leg electrode parts further comprises a pressure regulator configured to lower air pressure in a space between the adsorption plate, which adsorbs to the attachment part, and the attachment part.

7. The body composition measuring device of claim 1, wherein at least some of the arm electrode parts and the leg electrode parts comprises at least one electrode in contact with the attachment part and a sheet surrounding a periphery of the attachment part to maintain the contact.

8. The body composition measuring device of claim 1, wherein at least some of the arm electrode parts and the leg electrode parts comprises an electrode board configured to adjust a frequency of the electric current.

9. The body composition measuring device of claim 1, wherein
the body composition measuring device is in a form of a chair,
the arm electrode parts are disposed on arm rests of the chair, and
the leg electrode parts are disposed on parts of the chair in contact with the knees.

10. An operation method of a body composition measuring device, the method comprising:
measuring an impedance value of a measurement object by causing electric current to flow through different combinations of both hand electrode parts and both foot electrode parts, each of which has a plurality of electrodes, two arm electrode parts attached to elbow joint regions of arms, respectively, and two leg electrode parts attached to knee joint regions of legs, respectively; and
analyzing body composition of the measurement object, based on the impedance value of the measurement object.

11. The method of claim 10, wherein the measuring of the impedance value of the measurement object comprises causing electric current to flow through different combinations of the both hand electrode parts, the both foot electrode parts, the arm electrode parts, and the leg electrode parts, dividing the measurement object into nine segments of an upper left arm (ULA), a lower left arm (LLA), a upper right arm (URA), a lower right arm (LRA), a trunk (TR), an upper left leg (ULL), a lower left leg (LLL), an upper right leg (URL), and a lower right leg (LRL), and measuring the impedance values of the segments.

12. The method of claim 11, wherein the measuring of the impedance value of the measurement object comprises applying the electric current to first electrodes comprised in one pair among the both hand electrode parts, the both foot electrode parts, the arm electrode parts, and the leg electrode parts for a body composition measurement, obtaining a voltage for the electric current from second electrodes comprised in the pair, and measuring an impedance value of a segment between the pair by using the electric current and the voltage.

13. The method of claim 11, further comprising determining whether there is a segment determined to have edema among the segments based on a change in each impedance value of the segments.

14. The method of claim 10, wherein at least some of the arm electrode parts and the leg electrode parts comprises an adsorption plate adsorbing to an attachment part and at least one electrode in contact with the attachment part, inside the adsorption plate.

15. The method of claim 14, wherein the at least some of the arm electrode parts and the leg electrode parts further comprises a pressure regulator configured to lower air pressure in a space between the adsorption plate, which adsorbs to the attachment part, and the attachment part.

16. The method of claim 10, wherein the at least some of the arm electrode parts and the leg electrode parts comprises at least one electrode in contact with the attachment part and a sheet surrounding a periphery of the attachment part to maintain the contact.

17. The method of claim 10, wherein
the body composition measuring device is in the form of a chair,
the arm electrode parts are disposed on arm rests of the chair, and
the leg electrode parts are disposed on a part of the chair in contact with the knees.

18. A non-transitory computer-readable storage medium storing instructions that, when executed by a processor, cause the processor to perform the method of claim 10.
